(19) United States
(12) Patent Application Publication
(10) Pub. No.: US 2024/0140987 A1
(43) Pub. Date: May 2, 2024

(54) MICROBIAL ELECTROSYNTHESIS OF SINGLE CELL PROTEIN

(71) Applicant: Oregon State University, Corvallis, OR (US)

(72) Inventors: Hong Liu, Corvallis, OR (US); Luguang Wang, Corvallis, OR (US); Kevin Linowski, Corvallis, OR (US)

(73) Assignee: Oregon State University, Corvallis, OR (US)

(21) Appl. No.: 18/493,255

(22) Filed: Oct. 24, 2023

Related U.S. Application Data
(60) Provisional application No. 63/419,490, filed on Oct. 26, 2022.

(51) Int. Cl. C07K 1/24

(52) U.S. Cl. CPC C07K 1/24

(57) ABSTRACT

Single cell protein (SCP) is produced by applying a voltage to a microbial electrolysis cell (MEC) under anaerobic conditions for a period of time, whereby SCP is produced. The MEC includes a cathode comprising a hydrogen evolution reaction material, an anode comprising a biofilm on a carbon support, an electrolyte comprising carbon, nitrogen, and phosphorus, and a Methanococcus or Acetobacterium species in the electrolyte.

### CROSS-REFERENCE TO RELATED APPLICATION

This application claims the benefit of the earlier filing date of U.S. Provisional Application No. 63/419,490, filed Oct. 26, 2022, which is incorporated by reference herein in its entirety.

### ACKNOWLEDGMENT OF GOVERNMENT SUPPORT

This invention was made with government support under Grant No. 2014-38502-22598 awarded by the United States Department of Agriculture/National Institute of Food and Agriculture, and Grant No. EE0008844 awarded by the United States Department of Energy. The government has certain rights in the invention.

### FIELD

This disclosure concerns electrosynthesis of single cell protein using a microbial electrolysis cell.

### BACKGROUND

With the rapid increase of the global population and the limitations in animal feed production via industrial agriculture, the deficiency of protein will continue to rise. The highest edible protein yield by industrial agriculture is merely 40 g-soybean-protein/year/m²-land while the protein yield from beef is even lower at 2.2 g/year/m 2-land (Flachow sky et al., Animals, 7(3):25, 2017). Furthermore, the current agricultural land has already occupied over 30% of the global ice-free terrestrial surface, and 75% of the agricultural land is used for producing animal protein, indicating minimal space for improvement (Ramankutty et al., Annual Review of Plant Biology, Vol 69, 789-815, 2018). Besides the limited protein production capacity, there are many other limitations in the protein production through industrial agriculture, such as forest loss and fragmentation, greenhouse gas emissions, biodiversity destruction and high water use (Ramankutty et al., Annual Review of Plant Biology, Vol 69, 789-815, 2018). Single cell protein (SCP) is a promising supplement to conventional animal feed, as its production bypasses the disadvantages of animal feed production in industrial agriculture.

### SUMMARY

Microbial electrolysis cells (MECs) and use thereof for producing single cell protein (SCP) are disclosed. In some aspects, the MEC includes a cathode comprising a hydrogen evolution reaction material, an anode comprising a biofilm on a carbon support, an electrolyte comprising carbon, nitrogen, and phosphorus, and a Methanococcus or Acetobacterium species in the electrolyte. The MEC may further include a separator between the cathode and the anode. In any of the foregoing or following aspects, the MEC may be a stack MEC comprising a plurality of cathodes and a plurality of anodes.

A method for producing SCP may include applying a voltage of 0.6 V to 2.5 V from a power source to an MEC as disclosed herein under anaerobic conditions for a period of time, whereby SCP is produced. In some aspects, the MEC is operated at a temperature of 4° C. to 60° C. In any of the foregoing or following aspects, the method may further include periodically or continuously adding CO₂ to the electrolyte. The MEC may be operated in a batch, continuous, or semi-continuous mode.

In any of the foregoing or following aspects, the electrolyte may comprise an aqueous waste stream, an organic waste stream, or a combination thereof. In some aspects, the waste stream includes solids, and the method may further include removing at least a portion of the solids before introducing the electrolyte into the MEC. In certain implementations, at least a portion of the nitrogen, phosphorus, or nitrogen and phosphorus in the electrolyte is provided by a supplemental nitrogen source, a supplemental phosphorus source, or a combination thereof.

In any of the foregoing aspects, the electrolyte may include a Methanococcus species. In implementations where the Methanococcus species further synthesizes methane, the method may further include collecting the methane. In certain aspects, the Methanococcus species comprises Methanococcus maripaludis.

In any of the foregoing or following aspects, the SCP produced in the MEC may comprise all essential amino acids. In some aspects, the SCP comprises at least 35 wt % essential amino acids. The method may further include separating the SCP from the electrolyte. In some aspects, separating the SCP from the electrolyte comprises electrocoagulation, adding a coagulant to the electrolyte, or both electrocoagulation and adding a coagulant to the electrolyte. Separation of the SCP from the electrolyte may be performed in the MEC or downstream from the MEC.

In any of the foregoing aspects, the method may further include preparing the MEC for use by (i) inoculating the anode with the biofilm; (ii) placing a start-up medium in the MEC, the start-up medium comprising components suitable for growing microorganisms in the biofilm; (iii) operating the MEC under anaerobic conditions at an effective voltage and temperature for a period of time to grow the biofilm on the anode; (iv) replacing the start-up medium with the electrolyte; and (v) inoculating the electrolyte with the Methanococcus or Acetobacterium species.

### BRIEF DESCRIPTION OF THE DRAWINGS

FIG. 1 is a schematic view of a microbial electrolysis cell (MEC) including a biofilm-inoculated anode, a cathode, an electrolyte inoculated with a Methanococcus or Acetobacterium species, and a voltage source.

FIG. 2 is an exploded schematic view of a stacked electrode assembly, including two cathodes, two anodes, and three 3-D printed frames.

FIGS. 3A and 3B are graphs showing growth of planktonic cells estimated by optical density at 600 nm wavelength in Methanococcus maripaludis-inoculated MECs (M-MECs), control MECs (C-MECs), and Methanococcus pure culture controls (FIG. 3A), and current densities of the M-MECs and C-MECs (FIG. 3B). Error bars indicate the standard deviations of measurements from duplicate reactors.

FIGS. 4A and 4B are graphs showing hydrogen partial pressures in M-MECs, C-MECs and Methanococcus pure culture controls (FIG. 4A), and methane production volumes in M-MECs and Methanococcus pure culture controls (FIG. 4B). Error bars indicate the standard deviations of measurements from duplicate reactors.

FIG. 5 is a graph showing cathodic biogas recovery of the M-MECs during the operation for single-cell protein (SCP) production. Error bars indicate the standard deviations of measurements from duplicate reactors.

FIG. 6 is a graph showing total protein concentration changes in M-MECs, C-MECs and Methanococcus pure culture controls. Error bars indicate the standard deviations of measurements from duplicate reactors.

FIG. 7 shows the amino acid profile of harvested biomass in M-MECs. Error bars indicate the standard deviations of measurements from duplicate reactors.

FIG. 8 is a graph showing current density in an MEC including a nickel alloy mesh cathode.

### DETAILED DESCRIPTION

Single cell protein (SCP) produced by microorganisms is promising as a source of editable protein, for its high production rate and high protein content in dried biomass (Sharif et al., Aquaculture, 531:735885, 2021). The production of SCP also potentially bypasses the disadvantages of low production rate, high water footprint, high land use, and the environmental impacts. The existing approaches for SCP production are achieved through cultivating microalgae, fungi and bacteria (Jones et al., Current Opinion in Biotechnology, 61, 189-197, 2020). Bacteria contain the highest protein content (50-80%) in dried biomass and have demonstrated rapid growth (Ritala et al., Frontiers in Microbiology, 8, 2017). Their versatile metabolic pathways offer extensive possibilities for synthesizing SCP from various organic and inorganic substrates under aerobic and anaerobic conditions. Chemoautotrophic bacteria use inorganic substances (e.g., carbon dioxide) as a carbon source and for reduced compounds (e.g., hydrogen gas) as energy source without the dependence on light. The well-studied categories include acetogenesis, which converts carbon dioxide and hydrogen into acetate, and methanogenesis, which converts carbon dioxide and hydrogen into methane (Wang et al., International Journal of Hydrogen Energy, 43(29), 13064-13071, 2018; Wang et al., Bioresource Technology, 274, 557-560, 2019). The SCP production via chemoautotrophic growth of bacteria is promising for its sustainability, as the utilization of inorganic carbon could benefit the goal of carbon emission reduction. Also, metabolic products of the chemoautotrophic growth, such as methane, can be recovered as bioenergy or bioproducts. However, the key challenge for this SCP production approach lies in the renewable production and the utilization of hydrogen gas. Hydrogen gas is scarce in nature and most of the hydrogen produced today in the United States is via steam-methane reforming, which is not sustainable in the long term. Also, due to the low solubility of hydrogen, high pressure condition is often necessary to satisfy the bacteria for their growth, which could significantly increase the costs and difficulties for SCP production (Costa et al., Journal of Bacteriology, 195(7), 1456-1462, 2013; de Kok et al., Applied Microbiology and Biotechnology, 97(6), 2617-2625, 2013).

The microbial electrolysis cell (MEC) is a technology that can achieve complete degradation of organic waste streams for hydrogen production with the assist of electricity (Liu et al., Environmental Science & Technology 2005, 39(11), 4317-4320). Electroactive bacteria on the anodes degrade organics into carbon dioxide and protons while passing the electrons extracellularly to the cathode. With the assist of electricity, the abiotic cathode uses protons and electrons to synthesize hydrogen. The in situ generated biogas could be an ideal feedstock for SCP production from chemoautotrophic bacteria, as it could bypass the complex and expensive high pressure hydrogen storage, transportation, and sparging systems. Besides the superior hydrogen production performance, another unique feature of MECs is that the electroactive bacteria inhabit the electrode in the form of biofilms, leading to minimal presence of planktonic cells in the liquid phase. Such a feature allows MEC to be a promising SCP production platform for cultivating microorganisms with high purity.

Coupling a fast-growing hydrogen consuming organism with microbial electrolysis cells (MECs) could be a viable method for SCP production, potentially using organic agricultural and industrial waste streams and renewable electricity as the carbon and energy sources. The rapid in situ hydrogen production in MECs could significantly enhance the production rate of SCP. Some aspects of the disclosed MECs utilize a Methanococcus or Acetobacterium species in the electrolyte.

In some aspects, the MEC includes M. maripaludis as the source organism. The protein content in dried biomass is as high as 61% (Lupa et al., Applied and Environmental Microbiology, 74(21), 6584-6590, 2008). Besides tryptophan, which is undetectable using the analytical method, all other amino acids are abundant in M. maripaludis, indicating its nutrient efficacy is ideal (Goyal et al., Microbial Cell Factories, 14, article 146, 2015). In addition to using hydrogen as the electron donor for methane production and cell growth, M. maripaludis can directly accept electrons from a cathode. By removing hydrogen as the intermediate, the cell synthesis is more efficient. Thus, the coupling of MECs and M. maripaludis could potentially achieve the complete reuse of organic waste streams for SCP and methane production.

### I. Definitions

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximations unless the word “about” is recited.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise.

Definitions of common terms in chemistry may be found in Richard J. Lewis, Sr. (ed.), Hawley's Condensed Chemical Dictionary, published by John Wiley & Sons, Inc., 2016 (ISBN 978-1-118-13515-0).

In order to facilitate review of the various embodiments of the disclosure, the following explanations of specific terms are provided:

Anode: An electrode through which electric charge flows into a polarized electrical device. From an electrochemical point of view, negatively-charged anions move toward the anode and/or positively-charged cations move away from it to balance the electrons leaving via external circuitry.

Cathode: An electrode through which electric charge flows out of a polarized electrical device. From an electrochemical point of view, positively charged cations invariably move toward the cathode and/or negatively charged anions move away from it to balance the electrons arriving from external circuitry.

Coagulant: A compound or agent that results in particles coming together to form a flocculent mass. As used herein, the term “coagulant” refers to a compound or agent that results in single cell protein molecules in an electrolyte coming together to form a flocculent mass, which can be separated from the electrolyte.

Current density: A term referring to the amount of current per unit area. Current density is typically expressed in units of A/m².

Electrolyte: A substance containing free ions that behaves as an electrically conductive medium. Electrolytes generally comprise ions in a solution.

Essential amino acid: As used herein, the term “essential amino acid” refers to an amino acid that is not synthesized by mammals and is obtained through dietary intake. The nine essential amino acids are histidine, isoleucine, leucine, lysine, methionine, phenylalanine, threonine, tryptophan, and valine. Arginine is an essential amino acid for birds and certain mammals (e.g., cats, ferrets).

Hydrogen evolution reaction (HER): Catalytic production of hydrogen from water—2H⁺+2e⁻ →H₂. The HER reaction may provide an efficient way for environmental cleanup. For example, H₂ production using a microbial electrolysis cell (MEC) cleans wastewater as organic materials in the wastewater are degraded by microorganisms. Catalytic HER materials include, but are not limited to, platinum-group metals, certain nickel alloys, and molybdenum phosphide catalysts.

Microbial electrolysis cell (MEC): An electrolytic cell including a cathode and an anode, wherein the anode comprises a biofilm. The electrolyte may be an aqueous solution capable of sustaining the microorganisms comprising the biofilm. The electrolyte also may include microorganisms that are the same or different than those comprising the biofilm. M-MEC as used herein refers to an MEC inoculated with a Methanococcus species, such as M. maripaludis. C-MEC as used herein refers to a control MEC without a Methanococcus species inoculated in the electrolyte.

Single cell protein (SCP): Microbial protein obtained from cells of bacteria, yeast, fungi, or algae.

Volumetric current density: A term referring to the amount of current per unit volume. Volumetric current density is typically expressed in units of A/m³.

### II. Method for Producing Single Cell Protein

This disclosure concerns aspects of a method for producing single cell protein (SCP). SCP is produced by electrosynthesis using a microbial electrolysis cell (MEC).

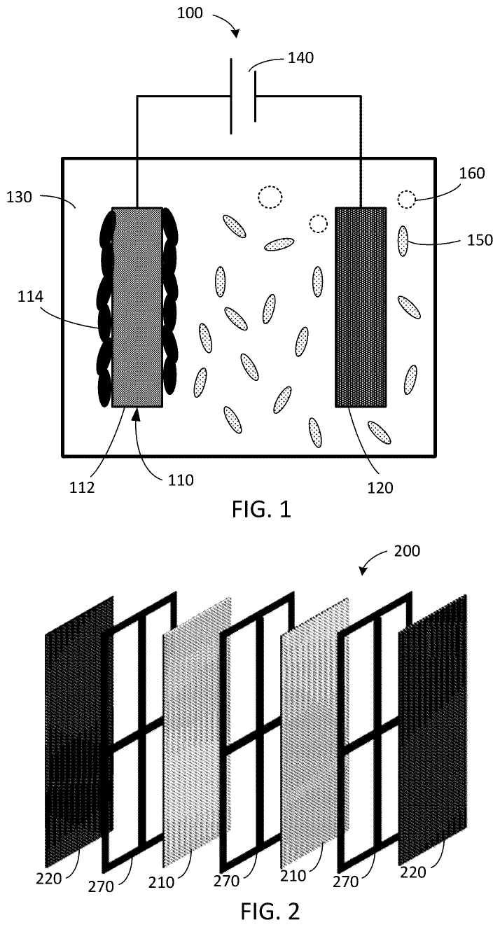

One exemplary MEC 100 is illustrated in FIG. 1. The MEC 100 comprises an anode 110, a cathode 120, and an electrolyte 130. In some aspects, the MEC 100 further comprises a voltage source 140 configured to be electrically coupled to the anode 110 and cathode 120. In certain aspects, the MEC 100 further comprises a separator (not shown) between the anode and cathode.

The anode 110 comprises a support 112 and a biofilm 114 deposited onto the support 112. In some implementations, the support 112 is a carbon support. In some examples, the support 112 comprises carbon cloth or carbon felt. In some aspects, the biofilm comprises electroactive bacteria.

In any of the foregoing or following aspects, the cathode 120 may comprise a hydrogen evolution reaction (HER) material or catalyst. Suitable HER materials and catalysts include, but are not limited to, nickel mesh, palladium nanoparticles, a molybdenum phosphide catalyst, a molybdenum sulfide (MoS₂) catalyst, a Co/FeCo catalyst, a NiMo/NiW catalyst, or an Fe/Fe₃C catalyst. In some aspects, the cathode 120 comprises a nickel mesh. The nickel mesh may comprise a nickel-copper alloy. In some implementations, the nickel-copper alloy comprises at least 63 wt % Ni and 28 wt % to 34 wt % Cu. In certain implementations, the nickel-copper alloy, prior to cycling the MEC, further comprises 0 wt % to 2.5 wt % Fe, 0 wt % to 2 wt % Mn, 0 wt % to 0.3 wt % C, and 0 wt % to 0.5 wt % Si. In some aspects, the cathode 120 comprises an HER catalyst applied to a support, such as a carbon-based material (e.g., carbon cloth) or stainless steel.

In any of the foregoing or following aspects, the electrolyte 130 may comprise carbon, nitrogen, and phosphorus. In some aspects, the electrolyte comprises an aqueous waste stream, an organic waste stream, or a combination thereof. In some examples, the waste stream may be a food or beverage industrial waste stream or an agriculture industry waste stream. In certain implementations, at least a portion of the nitrogen, phosphorus, or nitrogen and phosphorus in the electrolyte is provided by a supplemental nitrogen source, a supplemental phosphorus source, or a combination thereof. The supplemental nitrogen and/or phosphorus sources may be water-soluble nitrogen- and/or phosphorus-containing compounds, such as nitrate or phosphate salts, among others. The electrolyte 130 further comprises a Methanococcus or Acetobacterium species. Exemplary Methanococcus species include M. aeolicus, M. maripaludis, M. thermophila, M. vannielii, and M. voltae. Exemplary Acetobacterium species include A. bakii, A. carbinolicum, A. fimetarium, A. malicum, A. paludosum, A. tundrae, A. wieringae, and A. woodie. In some aspects, the electrolyte comprises a Methanococcus species. In certain aspects, the Methanococcus species is M. maripaludis. In any of the foregoing or following aspects, the electrolyte may have a pH of 5 to 9.5.

In any of the foregoing or following aspects, the MEC may be a stack MEC including a plurality of anodes and a plurality of cathodes. In one exemplary arrangement shown in FIG. 2, the stack MEC 200 includes two anodes 210 and two cathodes 220. The anodes 210 and cathodes 220 are in a spaced-apart arrangement. The stack MEC 200 may include spacers 270, such as 3-D printed frames, between adjacent anodes 210 and cathodes 220. Although the stack MEC 200 is illustrated with two anodes 210 and two cathodes 220, a person of ordinary skill in the art understands that a stack MEC may include any number of anodes and cathodes, such as 2-100 anodes and 2-100 cathodes, 2-50 anodes and 2-50 cathodes, 2-25 anode and 2-25 cathodes, 2-10 anodes and 2-10 cathodes, or 2-5 anodes and 2-5 cathodes. In one implementation, the stack MEC comprises alternating anodes and cathodes. In another implementation, the stack MEC comprises an arrangement of cathode, anode, anode, cathode, cathode, anode, anode, cathode, etc. Advantageously, the stack MEC may include equal numbers of anodes and cathodes. The stack MEC may further comprise separators between adjacent anodes or adjacent cathodes, and/or separators between adjacent anodes and cathodes.

The disclosed MECs may be used to produce SCP. With reference to FIG. 1, a method for producing SCP may include applying a voltage of 0.6 V to 2.5 V from a power source to the MEC 100 under anaerobic conditions for a period of time, whereby SCP 150 is produced. A voltage of 0.6 V to 2.5 V from a power source may correlate to a voltage of 0.6 V to 1.2 V measured at the electrodes of the MEC.

In any of the foregoing or following aspects, the MEC may be operated at a temperature of 4° C. to 60° C., such as a temperature within a range having endpoints selected from 4° C., 10° C., 15° C., 20° C., 25° C., 30° C., 35° C., 40° C., 45° C., 50° C., 55° C., or 60° C., wherein the range is inclusive of the endpoints. In some implementations, the MEC is operated at a temperature of 20° C. to 55° C.

In any of the foregoing or following aspects, the MEC may be operated at a current density of 10 A/m² to 80 A/m². In some aspects, the current density is within a range having endpoints selected from 10 A/m², 15 A/m², 20 A/m², 25 A/m², 30 A/m², 40 A/m², 50 A/m², 60 A/m², 70 A/m², or 80 A/m², wherein the range is inclusive of the endpoints.

In any of the foregoing or following aspects, a volumetric current density of 100 A/m³ to 6,000 A/m³. In some aspects, the volumetric current density is within a range having endpoints selected from 100 A/m³, 150 A/m³, 200 A/m³, 250 A/m³, 500 A/m³, 1,000 A/m³, 2,000 A/m³, 3,000 A/m³, 4,000 A/m³, 5,000 A/m³, or 6,000 A/m³, wherein the range is inclusive of the endpoints.

In any of the foregoing or following aspects, the MEC may be operated in a batch mode, a continuous mode, or a semi-continuous mode. In one implementation, the MEC is operated in a batch mode. In an independent implementation, the MEC is operated in a continuous or semi-continuous mode. In any of the foregoing or following aspects, the electrolyte may have a retention time of 4 hours to 48 hours in the MEC. In some aspects, the electrolyte has a retention time in range having endpoints selected from 4 hours, 6 hours, 8 hours, 12 hours, 18 hours, 24 hours, 30 hours, 36 hours, 42 hours, or 48 hours, wherein the range is inclusive of the endpoints.

The electrolyte 130 comprises carbon, nitrogen, phosphorus, and a Methanococcus or Acetobacterium species, as previously described. When the electrolyte includes a Methanococcus species, methane 160 may also be synthesized. In some aspects, the method further comprises collecting the synthesized methane. The methane may be collected by any suitable means. For example, generated methane gas may be vented from the MEC headspace and captured using a gas collection system. In any of the foregoing or following aspects, the Methanococcus species may be M. maipaludis. When the electrolyte includes an Acetobacterium species, acetate also may be synthesized. In such implementations, the method may further comprise using the acetate-rich liquid directly in other microbial processes for producing bioenergy and bioproducts. Alternatively, the acetate can be collected and further purified using methods such as precipitation, ion exchange, and/or evaporation.

In any of the foregoing or following aspects, the electrolyte may comprise an aqueous waste stream, an organic waste stream, or a combination thereof. Suitable waste streams may include waste streams from food and beverage industries, where the waste streams include carbon, nitrogen, and phosphorus. In some aspects, the aqueous waste stream, the organic waste stream, or the combination thereof comprises solids, the method further comprising removing at least a portion of the solids before introducing the electrolyte into the MEC. In some implementations, at least a portion of the nitrogen, phosphorus, or nitrogen and phosphorus in the electrolyte is provided by a supplemental nitrogen source, a supplemental phosphorus source, or a combination thereof. In certain aspects, the electrolyte has a pH of 5.0-9.5. In any of the foregoing or following aspects, the method may further comprise periodically or continuously adding CO₂ to the electrolyte. For example, CO₂ may be continuously added while operating the MEC.

In any of the foregoing or following aspects, the SCP may comprise all of the essential amino acids, i.e., amino acids that are not synthesized by mammals. In some implementations, the SCP comprises at least 35 wt % essential amino acids, such as at least 40 wt % essential amino acids. In some examples, the SCP comprises 35 wt % to 50 wt % essential amino acids, such as 35 wt % to 45 wt %, or 40 wt % to 45 wt % essential amino acids. Compared to poultry, meat, and fish, the abundance of essential amino acids is similar to that provided by fish, and is significantly higher than that found in poultry or meat. In some aspects, the SCP also is a significant source of limiting amino acids (lysine and methionine) found in plant-based animal feeds. In some examples, the SCP includes 5 wt % to 9 wt % lysine and 2 wt % to 4 wt % methionine. The SCP is suitable for many uses including, but not limited to, a supplement for use with conventional animal feed.

In some implementations, SCP is produced in the MEC at an average rate of 5 mg SCP/L electrolyte/h to 25 mg/L/h, such 10 mg/L/h to 20 mg/L/h, or 10 mg/L/h to 15 mg/L/h. In certain implementations, SCP is produced at an average rate of 250 mg SCP/m² electrode surface area/h to 1,000 mg/m²/h, such as from 300 mg/m²/h to 750 mg/m²/h, or 500 mg/m²/h to 600 mg/m²/h.

In any of the foregoing or following aspects, the method may further comprise separating the SCP from the electrolyte. In some aspects, separating the SCP from the electrolyte comprises electrocoagulation, adding a coagulant to the electrolyte, or both electrocoagulation and adding coagulant to the electrolyte. The coagulant may be any suitable water and/or wastewater coagulant. Exemplary coagulants include, but are not limited to, salts (e.g., calcium chloride, calcium oxide, ferric chloride, ferric sulfate, ferric chloride sulfate, ferrous sulfate, magnesium carbonate, sodium silicate, and the like) and organic coagulants (chitosan, alginate, starch, cellulose derivatives, gelatin, galactomannans, microbial polysaccharides, humic acids, tannins, etc.). In some aspects, the coagulant is aluminum-free. Electrocoagulation may be performed by any suitable method. For example, electrocoagulation may include using iron or other metals as plate, mesh or rod electrodes, and operating at a pH of 4-9.5 and a temperature of 10° C. to 50° C. with a retention time of 1 minute to 60 minutes. In some implementations, the SCP is flocculated by adding chitosan, such as from 1 mg/L to 50 mg/L chitosan at a pH of 4-8 with a contact time from five minutes to two hours. In any of the foregoing or following aspects, separation the MCP from the electrolyte may be performed in the MEC or downstream from the MEC.

In any of the foregoing or following aspects, the method may further include preparing the MEC for use. In some aspects, preparing the MEC includes (i) inoculating the anode with the biofilm; (ii) placing a start-up medium in the MEC, the start-up medium comprising components suitable for growing microorganisms in the biofilm; (iii) operating the MEC under anaerobic conditions at an effective voltage and temperature for a period of time to grow the biofilm on the anode; (iv) replacing the start-up medium with the electrolyte; and (v) inoculating the electrolyte with the Methanococcus or Acetobacterium species. In some implementations, the MEC is operated under anaerobic conditions until the current density reaches a stable plateau prior to replacing the start-up medium with the electrolyte. In any of the foregoing or following aspects, the effective voltage may be from 0.6 V to 1.8 V, and/or the temperature may be 4° C. to 60° C. In certain aspects, the voltage is 0.6 V to 1.8 V and the temperature is 20° C. to 55° C. The biofilm may be obtained from any suitable source. In some aspects, the biofilm is obtained from anodes of previously operated MECs. In other aspects, the anode biofilm may be obtained or enriched from wastewater sludge, anaerobic digester sludge, lake sediment, or soil. In any of the foregoing implementations, the start-up medium may comprise sodium acetate, sodium phosphate buffer, minerals, and vitamins. In certain examples, the start-up medium comprises 75 mM to 125 mM sodium acetate, 150 mM to 250 mM sodium phosphate buffer, minerals, and vitamins. The MEC headspace may be purged with an inert gas, such as nitrogen or argon to provide anaerobic conditions while growing the biofilm on the anode.

### III. Examples

### Materials and Methods

Reactor Design and Construction:

Single chamber MECs were constructed as the platform for SCP production via the cultivation of M. maripaludis. The single chamber MECs were made from medium bottles with a liquid volume of 100 mL and a headspace of 30 mL (Pyrex, Corning Inc, USA). The bottles were sealed using open top caps and modified butyl septum topped caps (VWR International, LLC). The cathode was fabricated using the procedure reported previously with a projected surface area of 12.5 cm 2 (Hu et al., Water Research, 42(15), 4172-4178, 2008). The catalyst for cathodic reaction was modified MoP, which was manufactured as previously described (Xie et al., ACS Catalysis, 9(9), 8712-8718, 2019). The anode was plain carbon cloth (Type-B, fuelcellearth.com) with a projected surface area of 12.5 cm 2. Each piece of the electrodes was secured by nylon screw and nuts, on a 3D-printed plastic frame. The electrode assembly was made by placing 2 pieces of the anodes between 2 pieces of the cathodes (FIG. 2). The electrodes were connected to a power supply using titanium wires.

Inoculation and Operation:

All MECs were inoculated by applying scraped biofilms from the anodes of MECs that had been operated for 6 months to the plain carbon cloth anodes of the MECs. The medium solution during the MEC start-up stage contains 100 mM sodium acetate, 200 mM sodium phosphate buffer, and necessary minerals and vitamins as reported previously (Hu et al., Water Research, 42(15), 4172-4178, 2008). Nitrogen gas was used to purge the reactor headspace to achieve an anaerobic condition. The MECs were operated with an applied voltage of 1.0 V at 32° C.

When the current density based on the projected surface area plateaued at >10 A/m², the medium solution was switched to medium solution for M. maripaludis, containing 200 mM morpholinepropanesulfonic acid (MOPS) buffer (pH 7), 100 mM sodium acetate, and other necessary components as described previously (Lie et al., Journal of Biological Chemistry, 280(7), 5236-5241, 2005). The incubation temperature was increased to 37° C. to benefit the growth of M. maripaludis. After 5 hours of operation to exclude possible residual oxygen, 5 mL of fresh growth medium containing active M. maripaludis was injected by a syringe into the MECs (M-MECs).

To investigate the effects of M. maripaludis inoculation on planktonic cell growth and SCP production, control MECs (C-MECs) were operated under the same condition without the inoculation of M. maripaludis. To further compare the growth of M. maripaludis between using in situ hydrogen from MECs and using ex situ hydrogen, additional control experiments were conducted by culturing M. maripaludis by supplementing ex situ hydrogen periodically in the same reactor in the absence of electrodes. All experiments were conducted in duplicates.

Analysis and Calculation:

The gas analysis was performed using the procedure described previously (Wang et al., International Journal of Hydrogen Energy, 43(29), 13064-13071, 2018). The growth of M. maripaludis was monitored by measuring the absorbance at 600 nm wavelength through using a spectrophotometer (UV-1700, Shimazu). The normalized absorbance was calculated by subtracting the absorbance measured before the inoculation from the actual absorbance. The absorbance measured before the inoculation is the result of a slight solution color change due to the possible discharge of cytochrome C from the anodic biofilms (Heitmann & Einsle, Biochemistry, 44(37), 12411-9, 2005). Voltage over a 1-ohm resistor was recorded using a multimeter with a data acquisition system (2700, Keithley) to calculate the current density based on the projected electrode surface area (Wang et al., International Journal of Hydrogen Energy, 43(29), 13064-13071, 2018). Cathodic hydrogen recovery was calculated by dividing the total electrons available in all the harvested hydrogen over the total electrons available in measured current. The weight of harvested biomass was estimated using a method suggested by the US EPA (Zhang et al., J Microbiol Methods, 94(3), 367-74, 2013). The electrons directed into cell synthesis were estimated based on the empirical formula of methanogenic biomass (C₅H₇O₂N) using the following equation:

5CO₂+10H₂+NH₃→C₅H₇O₂N+8H₂O

To analyze the total protein concentration, medium solution containing planktonic biomass (1 mL) was collected at each sampling point and frozen at −80° C. ethanol bath and thawed in a 42° C. water bath for 5 cycles, to complete cell lysis of the biomass (Bhaskar et al., Indian J Exp Biol, 43(3), 277-9, 2005). No obvious biofilm growth was observed on the cathode surface during the testing period. Total protein concentration was determined with the BCA Protein Assay Kit (Thermo Fisher Scientific, USA). To determine the amino acids profiling, the planktonic cells were harvested via centrifuge and hydrolyzed under acidic condition using the method described previously (Dai et al., Journal of Chromatography B—Analytical Technologies in the Biomedical and Life Sciences, 964, 116-127, 2014). The amino acids were analyzed by a high-performance liquid chromatograph (Agilent 1200 series), equipped with an AdvanceBio AAA column (Agilent, USA).

### Results and Discussion

Growth of Methanococcus maripaludis in Single Chamber MECs:

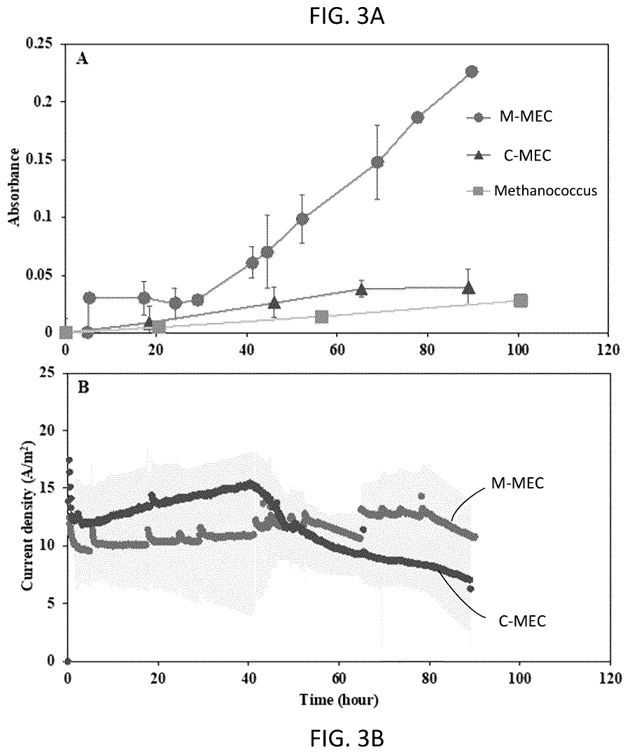

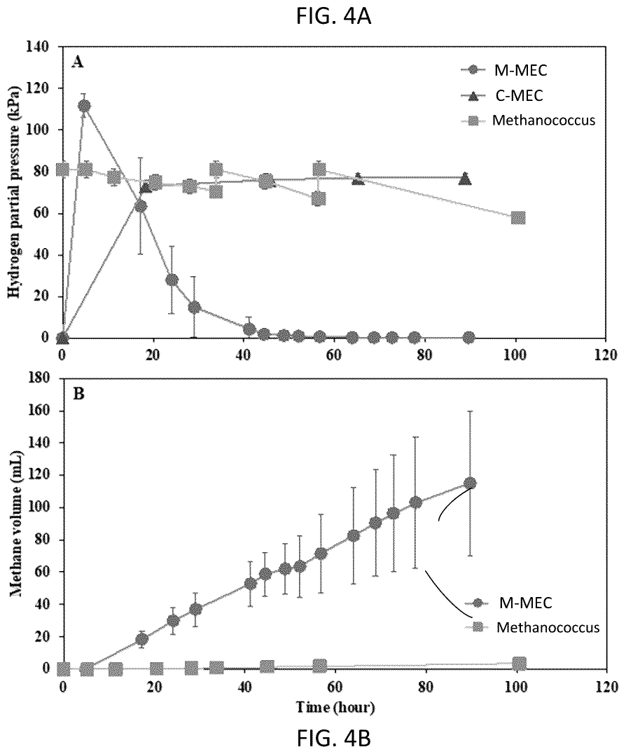

To examine the feasibility of coupling MEC with M. maripaludis to simulate the growth, the active growth culture was inoculated in the M-MECs at hour 5.3 of the batch operation. After a lag phase of approximately 24 hours, the optical density at 600 nm wavelength of the medium solution started to increase, as a result of M. maripaludis growth (FIG. 3A). The absorbance increased from 0.029 at hour 29.1 to 0.226 at hour 89.7, following a linear trend (R²=0.9964). The doubling time was measured as 11.2 h, corresponding to a specific growth rate of 0.062 h⁻¹. The average current density based on the electrode surface area from the duplicate reactors was also calculated. During the batch operation, the current density was maintained mostly between 10 to 15 A/m², corresponding to a theoretical maximum hydrogen production rate of 2.5 to 3.8 L/L/D (FIG. 3B). The decrease of current density for C-MEC after 40 hours was likely due to the depletion of substrate. The hydrogen partial pressure in the reactor headspace and methane production were shown in FIG. 4A. The hydrogen partial pressure demonstrated a sharp increase before the inoculation of M. maripaludis, attributed to the MEC process. After the inoculation of M. maripaludis, hydrogen partial pressure quickly decreased to nearly 0 at hour 48.9 and maintained at this low level for the rest of the batch operation. Methane production started immediately after the inoculation at hour 5.3, indicating the active growth of M. maripaludis (FIG. 4B).

While in the MEC-only controls (C-MECs), the optical density at 600 nm wavelength increased from 0 to 0.027 within a similar timespan of the M-MECs, corresponding to a doubling time of 31.8 h and a specific growth rate of 0.02211-1 (FIG. 3A). The increase of optical density could be attributed to the planktonic cells and/or cytochrome C discharged from the anodic biofilms. The average current density of the C-MECs was maintained at 8-15 A/m², which was similar to the M-MECs (FIG. 3B). The hydrogen partial pressure reached 73.0 kPa at hour 18.4 and maintained at 73.0 to 77.1 kPa throughout the batch (FIG. 4A). No methane was detected in the C-MECs. In the Methanococcus pure culture controls, the growth of M. maripaludis was significantly slower than that of M-MECs, with an estimated doubling time of 36.5 h and a specific growth rate of 0.019 h⁻¹ (FIG. 3A), despite that the hydrogen partial pressure was maintained at a much higher level (57.9 to 81.1 kPa), compared to the M-MECs (FIG. 4A). Methane production volume was also significantly lower than that of the M-MECs, indicating the growth of M. maripaludis using ex situ hydrogen was slower than the growth in M-MECs.

Compared to the shortest doubling time of M. maripaludis under optimal cultivation conditions with high pressure hydrogen (2.3 h), the growth of M. maripaludis in M-MECs was slower (Goyal et al., Microbial Cell Factories, 15, article 107, 2016). The increase of optical density during the active growth phase followed a linear regression, instead of the typical exponential regression. Considering that the production rate of hydrogen, which was reflected as current density in MECs, was quite constant during the operation, the linear increase of optical density indicated that hydrogen production rate was the limiting factor to the growth of M. maripaludis. The hydrogen production rate in the current MECs was estimated to be 2.5 to 3.8 L/L/D, with a relatively low electrode surface area to volume ratio of 50 m 2/m³ and volumetric current density of 250 to 380 A/m³. Improved hydrogen production rate was noted in a stack MEC (unpublished results) using the same electrode materials, but with a significantly improved surface area to volume ratio and operational conditions. The hydrogen production rate in the stack MEC reached 39.8 L/L/D, corresponding to a volumetric current density of 3948 A/m³. Based on the correlation between the growth rate and hydrogen production rate under substrate-limiting condition, the growth of M. maripaludis with significantly improved hydrogen production performance could reach the doubling time under optimal lab cultivation conditions (2.3 h).

During the active growth of M. maripaludis, hydrogen partial pressure was maintained at less than 5 kPa. Such a low hydrogen partial pressure could be the result of hydrogen consumption by M. maripaludis. However, M. maripaludis was reported to be electroactive, capable of accepting electrons directedly from electrodes for methanogenesis (Lohner et al., Isme Journal, 8(8), 1673-1681, 2014). Therefore, M. maripaludis could also directly utilize the electrons from the cathode and the protons and carbon dioxide from the anodic biological process for growth, leading to less or no hydrogen production from the cathodes. By removing hydrogen as the intermediate, such a process might offer an improved growth rate. However, during the relatively short testing duration in this study, no significant cathodic biofilm was observed. Thus, further study is warranted to investigate the electron-accepting mechanism of M. maripaludis in MECs for SCP production.

Electron Flux Analysis for SCP Production in M-MECs:

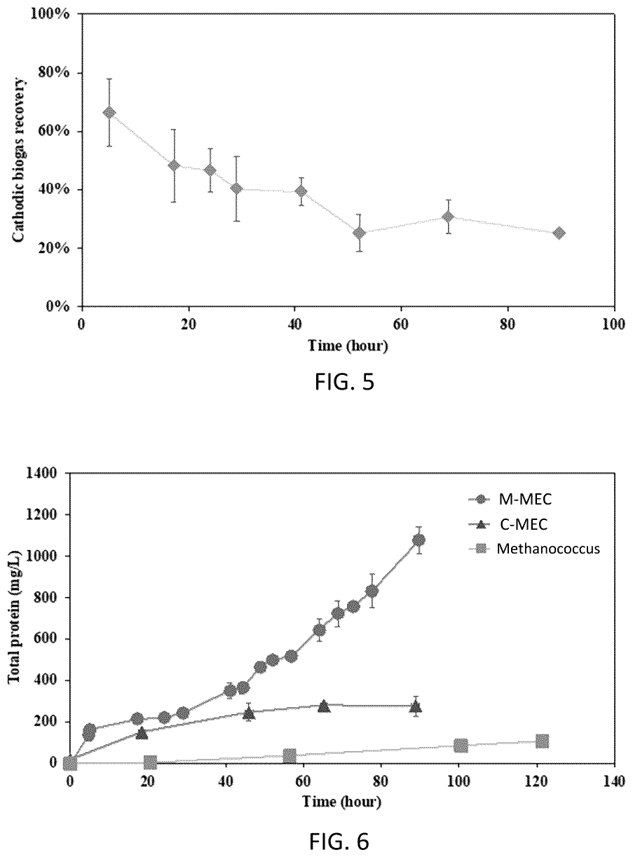

The electrons that flow to biogas (hydrogen and methane) were calculated and compared with the electrons in the electrical current to determine the cathodic biogas recovery. The cathodic biogas recovery followed a generally decreasing trend within the batch (FIG. 5). The highest electron recovery was 66.5% when M. maripaludis had not been inoculated. At 12 hours after the inoculation, electron recovery dropped to 48.4% and continued decreasing to 25.2% at the end of batch. During the batch operation, a total of 9047±1537 C electrons were transferred to the cathode. The electrons in recovered biogas, including both hydrogen and methane, were 3108±47 C, accounting for 34.4% of the total electrons. The electrons that were directed into cell synthesis was estimated to be 2835±5 C, indicating at least 31.3% electrons were utilized by the SCP synthesis. However, 34.3% of the electrons in the electrochemical process were not captured either in the produced biogas or the synthesized SCP. Before the inoculation of M. maripaludis, the relatively low cathodic biogas recovery (66.5%) also indicated that about 33.5% electrons were not directed into hydrogen production. A previous study suggested that homoacetogenic hydrogen consumption for acetate production could become significant under higher hydrogen partial pressures (20-35 kPa) (Wang et al., International Journal of Hydrogen Energy, 43(29), 13064-13071, 2018). Although the hydrogen partial pressure in this study was maintained at a low level after the inoculation of M. maripaludis, the rapidly produced hydrogen could still be partially consumed by the homoacetogenic bacteria, causing the internal recycle of electrons and lowered cathodic biogas recovery.

SCP Production Performance and Amino Acid Profiling:

To estimate the SCP production rate, total protein including both soluble and insoluble proteins in the solution was analyzed. At hour 5.3, a total protein concentration of 136 mg/L was detected, possibly as the result of discharging cytochrome C from the anodic biofilm (Heitmann & Einsle, Biochemistry, 44(37), 12411-9, 2005) (FIG. 6). After the lag phase, total protein concentration increased drastically from 220 mg/L (at hour 24.2) to 1077 mg/L (at hour 89.7). The total dried biomass at the end of the batch operation was 1.66±0.06 g/L. The trend of increase was consistent with the change of cell density demonstrated in FIG. 3A, indicating that the protein concentration increase was attributed to the growth of M. maripaludis. During the linear growth phase (hour 29.1 to 89.7), the total protein production rate was 13.7 mg/L/h. Given that the electrode surface area was possibly the limiting factor in the current reactor configuration, the total protein production per electrode surface area could provide a more explicit estimate, which was 548 mg/m²/h. In the C-MECs, the total protein concentration in the medium solution increased to 280 mg/L and plateaued at such a concentration (FIG. 6). In the Methanococcus pure culture controls, the total protein concentration constantly increased from 0 to 107 mg/L (FIG. 6). The average protein synthesis rates for the C-MECs and Methanococcus controls were 3.1 mg/L/h and 0.9 mg/L/h, respectively, which were significantly lower than the of the M-MECs (13.7 mg/L/h). The increases of total protein concentrations in both groups were also consistent with the cell density increases in FIG. 3A.

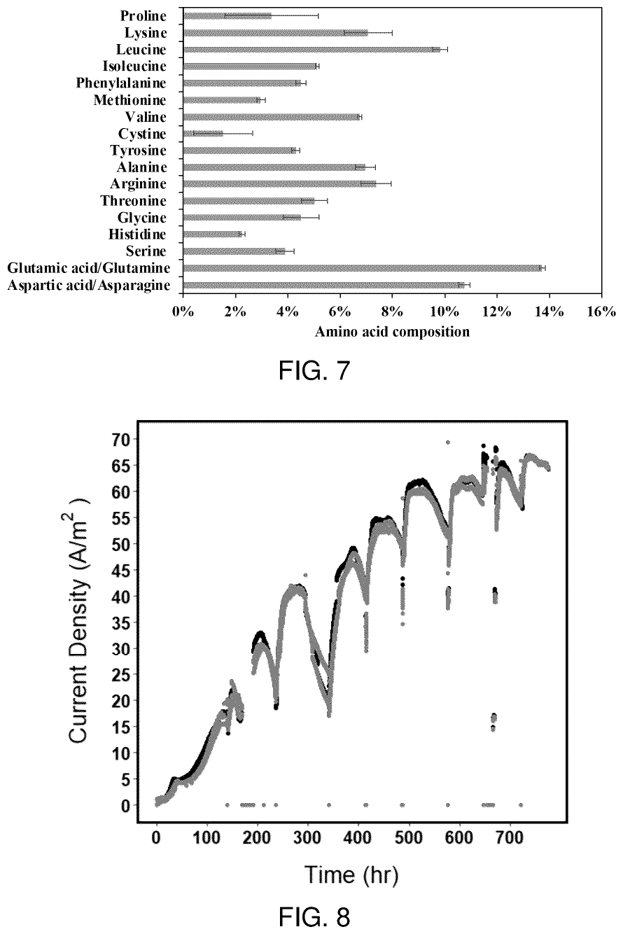

To further evaluate the nutrient efficacy of the harvested biomass, the amino acid profiling of the harvested protein from the M-MECs was analyzed (FIG. 7). Besides tryptophan, which is undetectable using the method in this study, the presence of all other amino acids was noted. The total abundance of essential amino acids was determined to be 43.5%, without including tryptophan. Compared to other common sources of dietary protein, such as poultry, meat and fish, the abundance of essential amino acids in SCP is close to that of fish (46.1%), without including tryptophan (Erkan et al., 2010). The abundance of essential amino acids in SCP is significantly higher than those of poultry and meat, which are 35.1% and 25.7%, respectively (Belhaj et al., ScientificWorldJournal, 2021, 6633774, 2021; Kim et al., Poultry Science, 92(11), 2844-2852, 2013). The abundance of essential amino acids in SCP is also significantly higher than that of the protein in plant-based animal feeds, which is less than 30% (Gorissen et al., Amino Acids, 50(12), 1685-1695, 2018). Lysine and methionine are known as the limiting amino acids in plant-based animal feeds, with abundances of less than 4% and less than 2% in many crop species, respectively (Gorissen et al., Amino Acids, 50(12), 1685-1695, 2018). The abundances of lysine and methionine in the harvested SCP are as high as 6.9% and 3.0%. Thus, SCP could serve as a promising complementary to plant-based animal feed, for the high content of essential amino acids and the high abundances of limiting amino acids.

Microbial SCP has been produced mostly through the cultivation of methane oxidizing bacteria (MOB) (Jones et al., Current Opinion in Biotechnology, 61, 189-197, 2020; Zha et al., Bioresour Technol, 320(Pt A), 124351, 2020). In a previous study using biogas derived from anaerobic digestions for cultivating methanotrophs, the specific growth rate ranged from 0.06 to 0.08 h⁻¹ (Zha et al., Bioresour Technol, 320(Pt A), 124351, 2020). The specific growth rate of methanogens in this study (0.062 h⁻¹) was similar to the growth rate of MOB. However, the cultivation of aerobic bacteria for SCP production is potentially limited by the higher energy demand for aeration, which could be avoided in the SCP production via MECs (Ritala et al., Frontiers in Microbiology, 8, 2017). Although the SCP production rate in this study was not as high as those of mixotrophic growth of microalgae and anaerobic growth of yeast, the disadvantages of these SCP production methods could be avoided. The operation of MECs does not require solar radiation and sterile conditions, which are necessary for the growth of microalgae (Jones et al., Current Opinion in Biotechnology, 61, 189-197, 2020). Compared to SCP production via anaerobic fermentation, the feedstock can be completely utilized for SCP production and biogas production, achieving waste valorization simultaneously (Jones et al., Current Opinion in Biotechnology, 61, 189-197, 2020; Liu et al., Environmental Science & Technology, 39(11), 4317-4320, 2005). Furthermore, in comparison with the protein production rate in industrial agriculture, the protein production rate in this study was 4800 g/m²-electrode/year, which is significantly higher than the highest protein production rate through growing soybeans (40 g/m²-land/year) (Flachowsky et al., Animals, 7(3):25, 2017). Besides, SCP production via MECs uses electricity as a major source of energy, which has become more available through sustainable production approaches (e.g. solar power). The spare electricity generated during peak production hours, which is hard to store, could be utilized for SCP production, benefiting the efficient usage of renewable electricity, and decreasing the energy cost for SCP production. Thus, SCP production via MECs could be a promising method for the sustainable and efficient production of protein, while connecting traditional agriculture with future energy systems.

Limitations and challenges cannot be neglected for the SCP production via MECs, compared to other SCP production methods. Although Methanococcus is the dominant species consuming hydrogen for growth in the M-MECs, biomass from other sources could potentially exist in the harvested biomass. One of the sources is the anodic biofilm, which might disperse planktonic cells into the solution (Cogan et al., mBio, 7(3), 2016). Another potential source is the growth of fermentative bacteria when using fermentable substrate in the MECs. Many syntrophic bacteria in the anodic community could scavenge cell debris for acetate production, enhancing current generation of the exoelectrogenic community (Gao et al., Bioresource Technology, 153, 245-253, 2014). When fermentable substrate (e.g. glucose) is available, the syntrophic bacteria could thrive as planktonic cells in the solution. The biomass from these sources could bring issues regarding the safety and the quality of the produced SCP. Furthermore, as a typical feature for fast growing organisms, the high nucleic acid content in the harvested microbial SCP could cause health issues and burdens to the downstream processes, when used as the main protein source in animal feed. However, these issues could be addressed through a two-chamber MEC design. Additionally, or alternatively, such an issue could be potentially resolved by mixing with plant-based animal feed, which is low in nucleic acid and certain amino acids that are high in microbial SCP (e.g. lysine and methionine) (Gorissen et al., Amino Acids, 50(12), 1685-1695, 2018). Thus, further investigation on SCP production via MECs is warranted to address the potential limitations and challenges on this technology.

Evaluation of Nickel Mesh Cathode:

An MEC with a MONEL 400 nickel alloy (available from McMaster-Carr, Atlanta, GA) mesh cathode and an electrolyte including M. maripaludis was evaluated. The cathode demonstrated high current density and stable performance over at least 700 hours (FIG. 8). The surface composition of the cathode was evaluated by X-ray photon spectroscopy before and after use for over 3,000 hours. The results are shown in Tables 1 and 2 below. The results show that the surface became enriched in Fe, Co, and Mn, and depleted in Cu and Ni after extended use. In addition to its use in a MEC for producing SCP, an MEC comprising the nickel mesh cathode also may be useful for hydrogen production and/or acetate synthesis (e.g., when the electrolyte includes an Acetobacterium species).

TABLE 1
Surface Composition of Fresh MONEL 400 Cathode

| Name | Peak BE | FWHM eV | Area (P) CPS.eV | Atomic % |
|---|---|---|---|---|
| P2p | 132.48 | 0.29 | 1440.23 | 0.47 |
| S2p | 168.53 | 1.37 | 5115.83 | 1.23 |
| N1s | 399.98 | 1.49 | 2895.76 | 0.91 |
| Cr2p | 587.58 | 0.03 | −119.25 | 0.00 |
| Mn2p | 651.93 | 0.08 | −316.64 | 0.00 |
| Fe2p | 712.43 | 1.59 | 14167.76 | 0.67 |
| Co2p | 791.18 | 0.00 | −2960.80 | 0.00 |
| Ni2p3 | 855.91 | 3.14 | 120854.50 | 6.71 |
| Cu2p3 | 932.85 | 1.74 | 93918.58 | 4.47 |
| Zn2p | 1019.20 | 0.00 | −1012.73 | 0.00 |
| Na1s | 1072.00 | 0.09 | 988.68 | 0.10 |
| Si2p | 102.09 | 1.28 | 2091.68 | 1.01 |
| C1s | 284.85 | 1.44 | 98812.91 | 48.05 |
| Ca2p | 347.41 | 1.67 | 8576.94 | 0.73 |
| Mo3d | 231.81 | 0.17 | 3701.23 | 0.16 |
| O1s | 531.61 | 1.92 | 176427.71 | 35.48 |

TABLE 2
Surface Composition of Aged MONEL 400 Cathode

| Name | Peak BE | FWHM eV | Area (P) CPS.eV | Atomic % |
|---|---|---|---|---|
| P2p | 139.40 | 0.15 | 1418.77 | 0.51 |
| S2p | 162.56 | 3.10 | 4146.53 | 1.10 |
| N1s | 399.99 | 2.32 | 12175.16 | 4.20 |
| Cr2p | 584.58 | 0.01 | −211.61 | 0.00 |
| Mn2p | 641.89 | 3.59 | 11975.99 | 0.68 |
| Fe2p | 711.82 | 5.34 | 92032.34 | 4.78 |
| Co2p | 781.99 | 4.04 | 94903.81 | 4.16 |
| Ni2p3 | 856.31 | 2.06 | 13032.03 | 0.80 |
| Cu2p | 953.23 | 0.00 | −1452.39 | 0.00 |
| Zn2p | 1021.86 | 2.15 | 9017.70 | 0.30 |
| Na1s | 1071.68 | 2.10 | 12653.25 | 1.39 |
| Si2p | 102.59 | 2.97 | 3600.34 | 1.92 |
| C1s | 285.07 | 2.43 | 82561.06 | 44.23 |
| Ca2p | 351.59 | 0.55 | 3199.86 | 0.30 |
| Mo3d | 232.36 | 1.87 | 5748.34 | 0.27 |
| O1s | 531.56 | 2.59 | 159534.78 | 35.34 |

### CONCLUSIONS

Rapid SCP production by coupling M. maripaludis and single chamber MECs was demonstrated in this study. The specific growth rate of M. maripaludis was 0.06211-1 with a doubling time of 11.2 h. The SCP production rate was 13.7 mg/L/h, which is comparable to other SCP production approaches, but bypassed the potential limitations in conventional methods, which are the dependence on solar radiation, high energy consumption, and low biomass yield. In the dried biomass, the weight of protein was over 60%. The electron flux analysis indicated that 31.3% electrons in the electrochemical systems were directed into SCP synthesis. The amino acid profiling revealed that the abundance of essential amino acids was as high as 43.5%. Besides tryptophan, which was undetectable using the analytical method, all amino acids are abundant. The results in this study suggested that such an approach could be promising for producing SCP as a supplement to conventional animal feed.

In view of the many possible embodiments to which the principles of the disclosure may be applied, it should be recognized that the illustrated embodiments are only preferred examples of the disclosure and should not be taken as limiting the scope of the invention. Rather, the scope of the disclosure is defined by the following claims. We therefore claim as our invention all that comes within the scope and spirit of these claims.

We claim:

1. A method for producing single cell protein (SCP), comprising:
applying a voltage of 0.6 V to 2.5 V from a power source to a microbial electrolysis cell (MEC) under anaerobic conditions for a period of time, whereby SCP is produced, the MEC comprising
a cathode comprising a hydrogen evolution reaction material;
an anode comprising a biofilm on a carbon support;
optionally, a separator between the cathode and the anode;
an electrolyte comprising carbon, nitrogen, and phosphorus; and
a Methanococcus or Acetobacterium species in the electrolyte.

2. The method of claim 1, wherein the electrolyte comprises a Methanococcus species.

3. The method of claim 2, wherein the Methanococcus species further synthesizes methane, the method further comprising collecting the methane.

4. The method of claim 2, wherein the Methanococcus species comprises Methanococcus maripaludis.

5. The method of claim 1, wherein:
(i) the SCP comprises all essential amino acids; or
(ii) the SCP comprises at least 35 wt % essential amino acids; or
(iii) both (i) and (ii).

6. The method of claim 1, wherein the MEC is operated at a temperature of 4° C. to 60° C.

7. The method of claim 6, further comprising periodically or continuously adding CO₂ to the electrolyte.

8. The method of claim 1, wherein the electrolyte comprises an aqueous waste stream, an organic waste stream, or a combination thereof.

9. The method of claim 8, wherein:
(i) the aqueous waste stream, the organic waste stream, or the combination thereof comprises solids, the method further comprising removing at least a portion of the solids before introducing the electrolyte into the MEC; or
(ii) wherein at least a portion of the nitrogen, phosphorus, or nitrogen and phosphorus in the electrolyte is provided by a supplemental nitrogen source, a supplemental phosphorus source, or a combination thereof; or
(iii) both (i) and (ii).

10. The method of claim 1, wherein the electrolyte has a pH of 5.0-9.5.

11. The method of claim 1, wherein the MEC is a stack MEC comprising a plurality of cathodes and a plurality of anodes.

12. The method of claim 1, wherein the MEC is operated at:
(i) a current density of 10 A/m² to 80 A/m²; or
(ii) a volumetric current density of 100 A/m³ to 6,000 A/m³; or
(iii) both (i) and (ii).

13. The method of claim 1, wherein the cathode comprises nickel mesh.

14. The method of claim 13, wherein the nickel mesh comprises a nickel-copper alloy comprising at least 63 wt % Ni and 28 wt % to 34 wt % Cu.

15. The method of claim 1, wherein the MEC is operated in batch mode.

16. The method of claim 1, wherein the MEC is operated in a continuous or semi-continuous mode.

17. The method of claim 16, wherein the electrolyte has a retention time of 4 hours to 48 hours in the MEC.

18. The method of claim 1, further comprising separating the SCP from the electrolyte.

19. The method of claim 18, wherein:
(i) separating the SCP from the electrolyte comprises electrocoagulation, adding a coagulant to the electrolyte, or both electrocoagulation and adding a coagulant to the electrolyte; or
(ii) separating the SCP from the electrolyte is performed in the MEC or downstream from the MEC; or
(iii) both (i) and (ii).

20. The method of claim 1, further comprising preparing the MEC by:
inoculating the anode with the biofilm;
placing a start-up medium in the MEC, the start-up medium comprising components suitable for growing microorganisms in the biofilm;
operating the MEC under anaerobic conditions at an effective voltage and temperature for a period of time to grow the biofilm on the anode;
replacing the start-up medium with the electrolyte; and
inoculating the electrolyte with the Methanococcus or Acetobacterium species.
